(19) United States
(12) Patent Application Publication
(10) Pub. No.: US 2025/0236874 A1
(43) Pub. Date: Jul. 24, 2025

(54) METABOLIC ENGINEERING OF ACTINOMYCETES BY SINGLE CELL MUTANT SELECTION

(71) Applicant: Turun yliopisto, Turun yliopisto (FI)

(72) Inventors: Mikko Metsä-Ketelä, Turun yliopisto (FI); Bikash Baral, Turun yliopisto (FI); Amirbehzad Akhgarinazarlou, Turun yliopisto (FI)

(21) Appl. No.: 18/697,961

(22) PCT Filed: Oct. 5, 2022
(86) PCT No.: PCT/FI2022/050664

§ 371 Date: Apr. 2, 2024

(30) Foreign Application Priority Data
Oct. 6, 2021 (FI) 20216030

(51) Int. Cl. C12N 15/76; C12N 9/04; C12N 9/10; C12N 9/12; C12N 13/00; C12P 17/06

(52) U.S. Cl. CPC C12N 15/76; C12N 9/0006; C12N 9/1077; C12N 9/1205; C12N 13/00; C12P 17/06; C12Y 101/03006; C12Y 204/02019

(57) ABSTRACT

The invention relates to a method for metabolic engineering of Actinomycetes. The method is based on traditional mutagenesis combined with reporter-guided single cell technologies. The method may be used for various purposes, such as for producing or increasing yields of endogenous target proteins or secondary metabolites, for replacing medium optimization in the production of target proteins or secondary metabolites, and for activating silent target genes or silent biosynthetic gene cluster. Also provided are metabolically engineered Actinomycetes strains obtainable by the method.

### CROSS-REFERENCE TO RELATED APPLICATIONS

This application is a national phase entry of, and claims priority to, International Patent Application No. PCT/FI2022/050664 (filed 5 Oct. 2022), which claims priority to Finnish Patent Application No. 20216030 (filed 6 Oct. 2021), the entire disclosures of which are incorporated herein by reference.

### FIELD OF THE INVENTION

The present invention belongs to the technical field of molecular biology. More specifically, the invention relates to a method for metabolic engineering of Actinomycetes for various purposes.

### BACKGROUND OF THE INVENTION

Actinomycetes are filamentous soil-dwelling Gram-positive bacteria that are widely utilized in diverse industrial biotechnology applications. Actinomycetes produce several important antibiotics, immunosuppressants, anticancer, antiparasitic and antifungal agents. Actinomycetes have remained a critical source of new drug candidates, since genome sequencing projects have identified a tremendous number of unknown biosynthetic gene clusters (BGC) that could encode novel secondary metabolites that can be used as drugs.

Actinomycetes also play an important role in soil ecology, where they have adapted to decompose complex organic plant and crustacean polymers. Proteins involved in these processes provide commercially available enzymes used particularly in paper and pulp (e.g. xylanases and cellulases) and detergent (e.g. lipases and amylases) manufacturing. Numerous enzyme applications can also be found in the food and beverage (e.g. proteases and glucose oxidase) and textile (e.g. pectinases and peroxidases) industries. Many medical diagnostic laboratories also depend on enzyme use in bioassays (e.g. cholesterol oxidase).

The manufacturing of both microbial natural products and proteins occurs in a closed environment in a bioreactor. Therefore, product yields are key determinants for the commercial viability of the manufacturing process. The yields of secondary metabolites are increased in strain development from few tens of milligrams per liter in wild type strains to several grams per liter in industrial production strains. Traditionally this decades long laborious process involves the generation of millions of mutants by random mutagenesis (UV, ethidium bromide, ethyl methanesulfonate) followed by individual cultivation of each generated mutant strain to assess production profiles and yields. Once the best producing mutant has been identified, the composition of medium ingredients needs to be optimized for high yield production. This represents another time-consuming challenge, since medium optimization is a multi-parameter problem, where modification of one component influences the ideal concentrations of the other medium ingredients.

Strain development has been improved by using reporter genes to identify mutants with increased transcription of target genes. For example, Xiang et al. (Metab. Eng. 11, 310-318 (2009)) have developed a double reporter system to screen improved clavulanic acid producing mutants. Guo et al. (Metab. Eng. 28, 134-142 (2015)) used the method for activation of silent jadomycin and gaudimycin BGCs. However, all current methods depend on detection of improved mutants on cultivation plates. This influences the throughput of the methodology, but also disassociates the screening conditions from the production environment in the bioreactor. Production profiles are highly dependent on growth conditions in Actinomycetes, and the best producing mutants on plates may not perform similarly under industrial settings. Thus, there is an identified need for advanced metabolic engineering techniques that overcome the problems associated with currently available techniques.

### SUMMARY

A method for metabolic engineering of Actinomycetes is disclosed. The method may comprise the following steps:
(i) identifying and selecting a promoter from a target gene or a gene involved in the biosynthesis of a target product,
(ii) cloning the promoter into a dual reporter construct to control the expression of a first reporter gene which is an antibiotic resistance gene and a second reporter gene encoding a fluorescent protein,
(iii) transforming an Actinomycetes strain with the reporter construct,
(iv) exposing the transformed Actinomycetes strain to conditions that induce random mutations, thereby producing a mutant library,
(v) transferring the mutant library into a liquid culture,
(vi) adding an antibiotic corresponding to the antibiotic resistance gene to the liquid culture to induce selective pressure to enrich mutants in which the transcriptional activity of the selected promoter has increased, thereby producing an enriched mutant library,
(vii) fragmenting mycelia in a manner by which bacterial cell walls remain intact in the enriched mutant library to enable screening by fluorescent cell sorting, and
(viii) screening the mutant library with fragmented mycelia by fluorescent cell sorting to obtain a metabolically engineered Actinomycetes strain that provides a fluorescent signal, wherein the extent of the fluorescent signal correlates with the expression level of the target gene or the gene involved in the biosynthesis of the target product.

Further aspects, embodiments and details are set forth in following figures, detailed description, examples, and claims.

### BRIEF DESCRIPTION OF THE DRAWINGS

The accompanying drawings, which are included to provide a further understanding of the invention and constitute a part of this specification, illustrate embodiments of the invention and together with the description help to explain the principles of the invention. In the drawings:

FIG. 1 is a schematic representation of the present single cell mutant selection (SCMS) platform for metabolic engineering. The strain improvement pipeline includes a, selection of promoter regions (P) from target gene(s), b, cloning the promoter into a reporter construct in E. coli and c, conjugation of the plasmid into the Actinomycetes production strain. d, Introduction of genome-wide disturbances by mutagenesis to generate overproducing strains. e, Transfer of the mutant library to a liquid culture and f, addition of antibiotics to impose selection to enrich positive mutants and reduce the size of the mutant library. g, Screening millions of mutants by FACS to find strains with highest fluorescence signal. Mutant libraries display a wide range of fluorescence values (x-axis) in comparison to a non-mutated negative control strain. Each dot represents a single cell. h, Verification of pure cultures by fluorometer analysis followed by more detailed i, enzymatic activity assays to find best performing j, protein overproduction strains or k, comparative production profile monitoring to find improved strains for l, production of microbial natural products.

FIGS. 2A to 2F illustrate improving ChoD protein production in S. laevendulae YAKB-15. FIG. 2A is a plasmid map for the positive control construct pS-GK containing the strong synthetic promoter SP44. Kanamycin resistance gene kan and sfGFP encoding superfolder Green Fluorescent Protein were used for selection and screening, respectively. Terminators T4 kurz25 and ECK12002960026 were used to insulate the promoter-probe construct. FIG. 2B shows the structure of the choD operon and selection of the promoter region. FIG. 2C shows the promoter for the choD operon ordered as synthetic DNA and cloned as XbaI-NdeI fragment to generate the activation construct pS_GK_ChoD. FIG. 2D shows cholesterol oxidase activity measurements from cultures grown in Y medium depicting increased yields of ChoD in three rounds of SCMS. Two mutants are shown for each round and the best performing mutant was selected for subsequent rounds of SCMS. FIG. 2E demonstrates preselection of best stains in second round mutagenesis after streak plating and fluorescence analysis of 12 pure cultures. FIG. 2F shows cholesterol oxidase activity measurements from cultures grown in GYM medium depicting increased yields of ChoD in three rounds of SCMS. Arrows depict the lineages of the strains in FIG. 2D and FIG. 2F. Significant differences were analyzed by one-way ANOVA with Tukey's post hoc analysis, and p<0.05 was considered statistically significant. ***p<0.001, **p<0.01, *p<0.05.

FIGS. 3A to 3F illustrate activation of a mutaxanthene BGC in A. orientalis NRRL F3213. FIG. 3A is a schematic representation of the organization of the aromatic type II polyketide BGC detected in A. orientalis NRRL F3213. The promoter targeted in SCMS is highlighted. FIG. 3D illustrates that FACS analysis of an enriched mutant library of A. orientalis NRRL F3213/pSGKP45 demonstrated enhanced sfGFP signal in a fraction of the mutants. Non-mutated A. orientalis NRRL F3213/pSGKP45 was used as a negative control, while A. orientalis NRRL F3213/pS-GK, where the fluorescence signal is generated through expression of sfGFP from a strong constitutive synthetic promoter SP44, was used as a positive control. Each dot represents a single cell. FIG. 3C shows an example of the phenotype of a GFP-positive mutant strain A. orientalis NRRL F3213/pSGKP45 UV1 (3) that produces pigmented metabolites in liquid cultures and on plates in contrast to the wild type strain. FIG. 3D is a schematic representation of the binding sites of primers used for detection of transcription from the core KS. gene by RT-PCR. FIG. 3E illustrates confirmation of cluster activation as detected by RT-PCR. Negative controls (lane 2 and 3) were reaction mixtures without reverse transcriptase enzyme and without RNA template, respectively. As a positive control, in vitro-transcribed human glyceraldehyde-3-phosphate dehydrogenase (GAPDH) control RNA was utilized (lane 4) that gave the product of size 496 bp. Detection of activated BGC on day 7 in A. orientalis NRRL F3213/pSGKP45 UV1 (3), where the expected 613 bp band is observed (lane 6). No products were observed in analysis of the wild type strain (lane 5) FIG. 3F shows time course analysis indicates that the BGC is activated on day 4 and transcription continues until day 8 in A. orientalis NRRL F3213/pSGKP45_UV1 (3).

FIGS. 4A to 4D illustrates structure elucidation, biosynthesis and yield improvement of mutaxanthenes. FIG. 4A shows analysis of culture extracts by HPLC-UV/Vis indicating the production of two additional metabolites 2 and 3 in A. orientalis NRRL F3213/pSGKP45 UV1 (3) in comparison to the wild type. The chromatogram traces were recorded at 256 nm. FIG. 4B shows chemical structures of mutaxanthenes A (2), B (3) and D (4) obtained from in A. orientalis NRRL F3213/pSGKP45_UV1 (3). FIG. 4C shows proposed biosynthetic pathway for mutaxanthenes. FIG. 4D illustrates analysis of production profiles of 20 second round GFP-positive mutants, which demonstrates increased yields of 2 and 4. Peak areas for the two metabolites were combined to reflect the total carbon flux to the activated pathway.

### DETAILED DESCRIPTION

### Definitions

Unless otherwise defined, the terms and expressions used in this specification and claims have the meanings generally applicable in the field of molecular engineering. Some of the terms and expressions used herein are have the meanings defined herein below. Further definitions may appear later in the specification.

As used herein, the singular expressions “a”, “an” and “the” mean one or more. Thus, a singular noun, unless otherwise specified, carries also the meaning of the corresponding plural noun.

The term “and/or” in a phase such as “X and/or Y” shall be understood to mean either “X and Y” or “X or Y” and shall be taken to provide explicit support for both meanings or for either meaning.

As used herein, the term “Actinomycetales” refers to an order of Actinobacteria. Members of Actinomycetales are often called Actinomycetes. They are Gram-positive and generally grow as colonies that resemble mycelia of fungi.

As used herein, the term “Streptomyces” refers to a genus of Actinomycetes. They are Gram-positive and aerobic. Members of the genus Streptomyces contribute to almost 70-80% of known secondary metabolites, many Streptomyces being antibiotic producers. Non-limiting examples of Streptomyces include S. lavendulae, S. aureofaciens, S. rimosis, S. griseus, S. peucetius, S. galilaeus, S. coelicolor, S. albus, S. hygroscopicus, S. avermitilis, S. kanamyceticus and S. venezuelae.

As used herein, “Amycolatopsis” refers to another genus of Actinomycetes. The genus Amycolatopsis is of special importance for its capacity to produce several commercially and medicinally important antibiotics, other secondary metabolites such as immuno-suppressants and anti-cancer agents to name a few examples. Non-limiting examples of Amycolatopsis include A. orientalis, such as A. orientalis NRRL F3213, and A. mediterranei.

As used herein, the term “target gene” refers to any endogenous bacterial gene whose expression is to be activated or enhanced by the present method. Preferably, the target gene codes for the target protein directly or is involved in the biosynthesis of the desired secondary metabolite.

As used herein, the term “target product” refers to any organic compound, such as a protein or a secondary metabolite, whose synthesis in a bacterial host is to be activated or yield increased by the present method.

As used herein, the term “secondary metabolite” refers to organic small molecule compounds produced by bacteria which are not directly involved in the normal growth, development, or reproduction of the organism. Secondary metabolites often have diverse, unusual and complex structures, and include compounds such as antibiotics, toxins, immunosuppressants and anticancer agents.

As used herein, the term “biosynthetic gene cluster” (BGC) refers to a locally clustered group of two or more genes that together encode a biosynthetic pathway for the production of a secondary metabolite.

As used herein, the term “unknown BGC” refers to a previously unknown BGC, whereas the term “silent BGC” refers to a BGC that is not actively expressed, or only lowly-expressed in a manner where the product of the pathway cannot be detected from culture extracts, under standard laboratory growth conditions. Since these unknown and silent or cryptic BGCs greatly outnumber the constitutively active ones, methods that reliably awaken them provide a powerful tool to increase the reservoir of potentially therapeutic small molecules and proteins.

As used herein, the term “endogenous” refers to an entity, such as a target gene or a target product, that already exists as a component of a cell, or as a component capable of being produced by a cell. As used herein, the entity may be naturally endogenous or obtained via earlier genetic modification of the host.

As used herein, the term “promoter” refers to a regulatory element formed by a DNA sequence which is required for the expression of a gene operably linked to the promoter. The promoter acts as the binding site for an RNA polymerase to initiate transcription of the gene. In the context of the present invention, the promoter is preferably a promoter of a target gene or a promoter involved in the synthesis of a target product.

As used herein, the term “operably linked” refers to a relationship between two or more components that allows them to function in an intended manner. For example, where a reporter gene is operably linked to a promoter, the promoter actuates the transcription, and hence expression, of the reporter gene.

As used herein, the term “terminator sequence” refers generally to a DNA sequence that signals termination of transcription to an RNA Polymerase. In the context of the present invention, a reporter cassette can be insulated by placing a terminator sequence in front of a promoter. In this way promoter leakage due to transcription from upstream genes can be avoided. Both synthetic and natural terminators are known in the art and can be employed in the present invention as desired. T4 kurz23 is a non-limiting example of available synthetic terminator sequences, whereas ECK12002960024A is a non-limiting example of available natural terminators.

As used herein, the term “reporter cassette” refers broadly to a polynucleotide comprising one or more reporter genes that are operably linked to a promoter. Optionally, a reporter cassette may comprise a terminator sequence located in front of the promoter. Moreover, a reporter cassette may comprise any appropriate ribosome binding sites.

As used herein, the term “ribosome binding site” refers to a sequence that is located upstream of the start codon in transcribed mRNA and responsible for the recruitment of a ribosome during the initiation of translation.

As used herein, the term “reporter construct” refers to a vector comprising a reporter cassette. For bacterial transformation, the vector may be, for example, a plasmid, a bacteriophage, a phagemid or a cosmid. As the most commonly used form of a vector, the term “plasmid” refers to circular double-stranded DNA wherein an additional DNA fragment, such as a reporter cassette can be inserted.

As used herein, the term “dual reporter construct” refers to a vector comprising a reporter cassette, wherein two different reporter genes are operably linked to a promoter of a target gene or a promoter of a gene involved in the synthesis of a target product.

As used herein, the term “reporter gene” refers to a gene expressing or involved in the expression of a marker which can be easily detected and which is suitable for distinguishing hosts that contain the reporter construct. Hosts that do not contain the reporter construct or do not express the reporter genes of the reporter construct do not provide the reporter signal. For example, the reporter gene may be an “antibiotic resistance gene” endowing the host with antibiotic resistance, so that it is capable of surviving on nutrient media on which other hosts, which do not contain or express the reporter construct, will not continue to divide and eventually die. Another particular example of a suitable reporter gene is a gene that encodes a color-forming protein, such as a fluorescent protein.

As used herein, the term “selection marker” refers to a gene expressing or involved in the expression of a selection marker which can be easily detect-ed and which is suitable for distinguishing hosts that contain the reporter construct from hosts that do not contain the reporter construct. Preferably, the selection marker is an “antibiotic resistance gene” endowing the host with antibiotic resistance, so that it is capable of surviving on nutrient media on which other hosts, which do not contain or express the reporter construct, will not continue to divide and eventually die.

As used herein, the term “transform” or “transformation” refers broadly to the transfer of a nucleic acid molecule, such as a reporter construct, into a host bacterial cell. Host bacterial cells comprising the transformed nucleic acid fragment are referred to as “recombinant” or “transgenic” or “transformed” organisms. For transformation, the bacterial host cells need to be competent, i.e. able to take up foreign DNA. Protocols for preparing competent cells are readily available in the art. Competent bacterial host cells are also commercially available from different sources. Some bacteria may be naturally competent. Transformation may be achieved by any appropriate technique available in the art including, but not limited to, those involving heat shock, electroporation, conjugation or active or passive targeting for example by means of liposomes or nanoparticles as transfection agents.

As used herein, the term “mutation” refers to a substitution of a residue within a sequence, e.g. a nucleic acid or amino acid sequence, with another residue, or a deletion or insertion of one or more residues within a sequence, whereas the term “random mutagenesis” refers to the formation of mutations in a random fashion. As well known in the art, random mutagenesis can be induced by exposing cells to mutagens, i.e. chemical compounds or forms of radiation that cause irreversible mutations. Non-limiting examples of chemical mutagens include base analogs such as bromouracil and aminopurine, alkylating agents such as methanesulfonate, intercalating agents such as ethidium bromide, and metal ions such as nickel, chromium, cobalt, cadmium, arsenic, chromium and iron ions. Non-limiting examples of physical mutagens include radiation such as ultraviolet (UV) light, X-rays, alpha rays, neutrons, and other ionizing and non-ionizing radiations.

As used herein, the term “mutation library” refers to a repertoire of mutated bacterial cells that carry a dual report construct.

As used herein, the term “liquid culture” refers to a suspension of growing bacterial cells in a nutrient solution or broth.

As used herein, the term “single cell” in the expressions such as “single cell selection” does not refer to single cells stricto sensu but also to clusters of a small number of cells, such as clusters of some cells, usually but not limited to two, three or four cells.

As used herein, the term “mycelium” refers to a fungus-like bacterial colony that grows as a network of long filaments.

As used herein, the term “mutaxhantene” refers to a member of a polyketide family of antibiotics. At least five mutaxanthenes are known to date, designated as mutaxanthenes A-E.

### Description

The present invention provides a method for metabolic engineering of Actinomycetes. In the method, reporter-guided single cell technologies are combined to traditional mutagenesis to increase the efficiency of metabolic engineering. Use of a double reporter system that allows selection of positive mutants from liquid cultures and screening of outstanding cells by fluorescence-activated cell sorting (FACS).

In the first step of the method, a promoter region of a target gene or of a gene involved in the biosynthesis of a target product is to be identified and selected. Means and methods for the identification and selection are readily available in the art including, for example, predictive algorithms and manual curation of genome sequences. If the promoter for a target gene or a gene involved in the biosynthesis of a target protein is already known, it is to be understood that employing the known promoter sequence qualifies as the step of identifying and selecting a promoter region from the target gene or the gene involved in the biosynthesis of the target product.

Once a promoter region from a target gene or a gene involved in the synthesis of a target product has been identified and selected, it is to be cloned into a dual reporter construct to control the expression of two different reporter genes, preferably one being an antibiotic resistance gene and the other being a gene encoding a fluorescent protein.

Gene sequences that impart antibiotic resistance are readily available in the art. The antibiotic against which resistance is imparted by the gene sequence can be, for example, selected from the group comprising ampicillin, tetracycline, kanamycin, chloramphenicol, spectinomycin, hygromycin, sulphonamide, trimethoprim, bleomycin/phleomycin, gentamicin and blasticidin. However, preferred antibiotic resistance genes in the dual reporter construct as reporter genes are those that confer antibiotic resistance by encoding enzymes, such as aminoglycoside acyltransferases or hygromycin phosphotransferase, that inactivate their target antibiotics, as opposed to genes that confer antibiotic resistance by encoding enzymes that modify their target antibiotics. This allows linking the transcription level of the antibiotic resistance gene to the survival of the host strain under elevated concentrations of the antibiotic in question. In other words, only hosts with a higher transcriptional activity of the resistance gene survive in higher concentrations of the antibiotic, while hosts with a wider range of transcriptional activity survive in lower concentrations of the antibiotic. Thus, by adjusting the selective pressure by modifying the concentration of the antibiotic in question, it is possible to achieve host strains with a desired level of transcriptional activity of the resistance gene, correlating with the promoter activity of the dual reporter construct, which in turn correlates with the expression level of the target gene or yield of the target product. Non-limiting examples of preferred antibiotic resistance genes to be used as reporter genes in the dual reporter construct include kan and hyg, which confer resistance to kanamycin and hygromycin, respectively, through inactivation thereof.

Gene sequences that encode fluorescent proteins are readily available in the art. The fluorescent protein may be a one selected from the group consisting of green fluorescent protein (GFP), red fluorescent protein (RFP), yellow fluorescent protein (YFP), cyan fluorescent protein (CFP), and orange fluorescent protein (OFP), but is not limited thereto. Non-limiting examples of GFPs include a superfolder green fluorescent protein (sfGFP), an enhanced green fluorescent protein (eGFP), and emerald GFP. Non-limiting examples of RFPs include monomeric red fluorescent protein (mRFP), mCherry, tdTomato, mStrawberry, J-red, and DsRed. Non-limiting examples of YFPs include Venus, mCitrine, YPet, and eYFP. Non-limiting examples of CFPs include CyPet, mCFPm, and Cerulean. Non-limiting examples of OFPs include mOrange and mKO.

Methods well known to those skilled in the art can be used to construct a dual reporter construct described herein. One such method involves ligation via cohesive ends. Compatible cohesive ends can be generated on a DNA fragment to be cloned and a selected vector by the action of suitable restriction enzymes. These ends will rapidly anneal through complementary base pairing and any remaining nicks can be closed by the action of a DNA ligase.

If a DNA fragment to be cloned does not contain suitable restrictions sites, such sites can be created by known methods. For example, DNA fragments with non-compatible protruding 3′ termini can be made blunt by bacteriophage T4 DNA polymerase or E. coli DNA polymerase I which remove the protruding 3′ termini and fill in recessed 3′ ends. Synthetic linkers, pieces of blunt-ended double-stranded DNA which contain recognition sequences for appropriate restriction enzymes, can then be ligated to blunt-ended DNA fragments by T4 DNA ligase. They can be subsequently digested with the appropriate restriction enzymes to create desired cohesive ends and ligated to a vector with compatible termini. It is also possible to use adaptors, chemically synthesised DNA fragments which contain one blunt end used for ligation but which also possess one preformed cohesive end. A variety of synthetic linkers and adaptors are commercially available from a number of sources.

It is also envisaged that a dual reporter construct to be employed in the present invention can be synthetized by means and methods readily available in the art. Such a synthetic construct can be ordered from various commercial sources.

In some embodiments, various bacterial hosts, such as E. coli, may be used to propagate the dual reporter construct prior to transformation into an Actinomycetes strain. To this end, the dual reporter construct should further comprise a selection marker operably linked to an active promoter to enable selection of those propagation host cells that contain the dual reporter construct from those propagation host cells that do not contain the dual reporter construct. In preferred embodiments, the selection marker is an antibiotic resistance gene. Notably, the antibiotic resistance gene can be any antibiotic resistance gene irrespective of its mode of action. However, the antibiotic resistance gene to be used as a selection marker must be different from the antibiotic resistance gene to be used as a reporter gene. Furthermore, the selection marker and reporter antibiotic resistance genes should not provide cross-resistance to the used antibiotics.

After cloning, a desired host strain of Actinomycetes is transformed with the present dual reporter construct by any suitable technique available in the art. If a bacterial host other than the recipient Actinomycetes strain, e.g. E. coli, is used for propagating the dual reported construct, conjugation is a convenient transformation technique. In conjugation, the propagation host transfers the dual reporter construct to the recipient Actinomycetes through a direct contact. This can be achieved by culturing the propagation host cells and the recipient cells in the same culture under conditions known in the art.

Next, a mutant library is produced by exposing the transformed Actinomycetes strain to conditions that induce random mutations.

The mutant library is then transferred into a liquid culture in appropriate nutrient medium. The same antibiotic against which the antibiotic resistance gene endows resistance is then added into the liquid culture nutrient medium. Consequently, cells expressing the antibiotic resistance gene can be selected from those not expressing it. In other words, cells that express the antibiotic resistance gene exhibit antibiotic resistance thus being capable of surviving in nutrient medium comprising the antibiotic, whereas those cells that do not express the reporter gene die. Selective pressure can be adjusted by varying the amount of the antibiotic in the nutrient medium. In this way it is possible to enrich those cells in which the transcriptional activity of the selected promoter has increased. Notably, increased promoter activity is indicative of increased expression of the target gene or yield of the target product.

Ultra-high throughput screening is next utilized to identify single cells from the enriched mutant library with the highest promoter activity. This is done using the second reporter gene, namely a gene encoding a fluorescent protein, and single cell selection by fluorescent cell sorting. This is in contrast to prior art methods employing reporter-guided mutant selection, wherein mutant libraries are exposed to the selective pressure by antibiotics and further selected based on the expression of the second reporter gene, usually a gene encoding a color-forming protein, on culture plates, adversely affecting throughput of the method.

FACS is a technology for cell sorting by employing flow cytometry. FACS rapidly detects the particles or cells in a liquid state when they pass a sensing point, and measures the different characteristics of each cell concurrently (size of the cell, internal composition of the cell, and functions of the cell), and depending on the cases, it can select and sort specific cells. For example, if a fluorescent protein-encoding gene is used as a reporter gene, the cells expressing the reporter gene or the cells not expressing the reporter gene can be detected and sorted accordingly.

However, since mycelia of growing Actinomycetes strains are generally orders of magnitude larger than the nozzle size of FACS equipment, it is not possible to analyse the enriched mutant library directly by FACS. Surprisingly, this problem can be overcome by fragmenting the mycelium of individual mutants present in the library to achieve a single cell suspension or a suspension with clusters of only few cells, which reduces the particle size in such a manner that the cells can be passed through the fluorescent cell sorting instrument. Any available fragmentation technique may be employed provided that the bacterial cell walls remain intact, thus keeping the cells viable and enabling culturing of the cells for any desired purpose, such as for the production of the target product and/or for additional rounds of mutagenesis and single cell mutant selection. A preferred technique for mycelial fragmentation is sonication, a process of applying sound energy to agitate particles in a liquid.

Ultrasonication is one way of performing sonication. Accordingly, the term “ultrasonication” refers to a sonication technique that employs ultrasound. As used herein, the term “ultrasound” refers to all acoustic energy with a frequency above human hearing (20,000 hertz or 20 kilohertz). Both the amplitude and the pulse time of the applied ultrasonication may vary. Those skilled in the art are able to choose appropriate sonication conditions (e.g., the amplitude and the pulse time) such that the bacterial cell walls remain intact. Typically, the amplitude is around 20 and the pulse time within the range of 3-30 s, for example 3-25 s, 5-25 s, 3-10 s, 5-10 s or 3-5 s.

Notably, techniques (e.g. lysozyme treatment) that yield bacterial protoplasts, i.e. bacterial cells without the cell wall are not suitable for use in the present method to enable fluorescent cell sorting. This is because, although protoplasts of Actinomycetes could be sorted by single cell fluorescent sorting such as FACS, such cells are fragile and have low survival rates, which, therefore, make them non-ideal for single cell mutant selection. Accordingly, the terms “mutant library” or “enriched mutant library” do not refer to libraries of mutated protoplasts. Moreover, since protoplasts do not have cell walls, lysozyme treatment is not encompassed by the term “fragmenting mycelia in a manner by which bacterial cell walls remain intact”.

By screening the sonicated mutant library based on the extent of a fluorescent signal produced by individual cells, it is possible to sort and collect those metabolically engineered Actinomycetes cells that are likely to be the best producers of the target product. This is because the extent of the fluorescent signal correlates with the expression level of the target gene or the production rate or yield of the target product.

As indicated above, the present method of metabolic engineering may comprise any desired number of repeated rounds of random mutagenesis and subsequent single cell mutant selection, thus enabling continued metabolic engineering of the selected Actinomycetes strain.

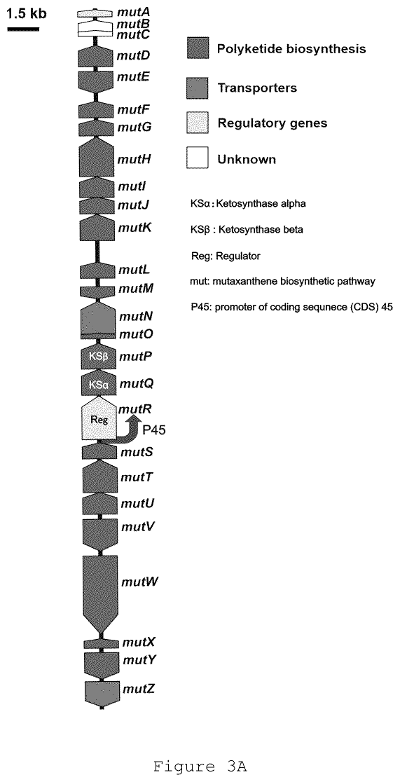

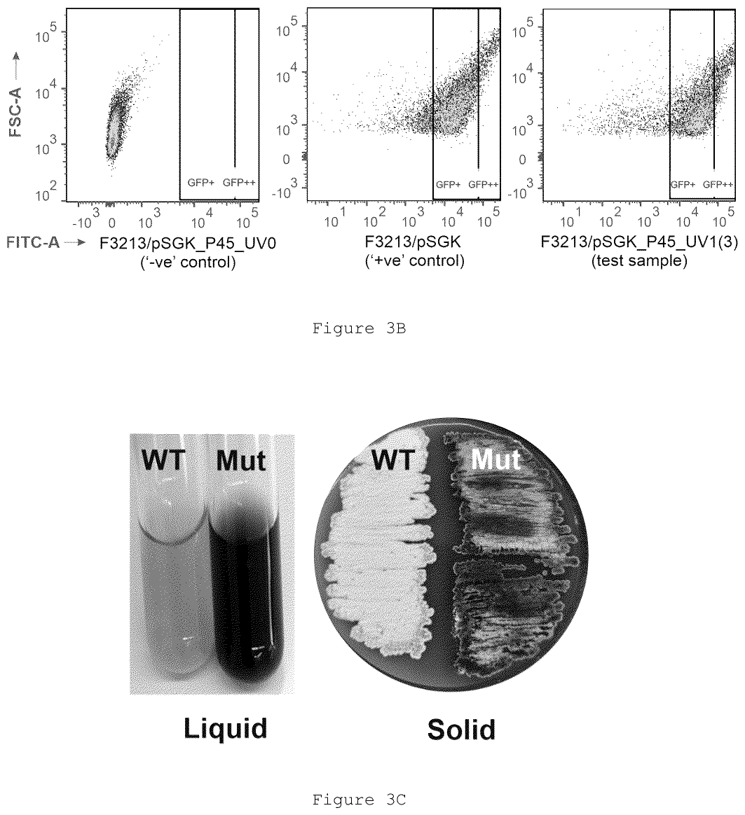

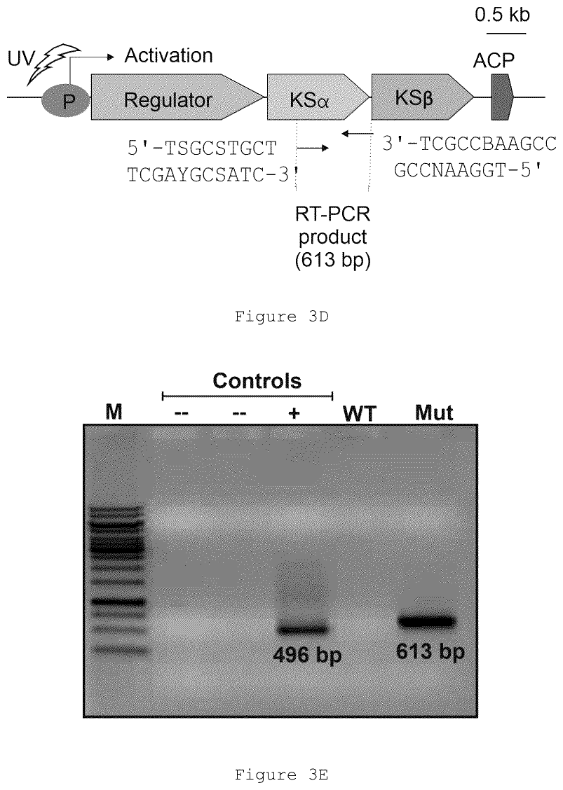

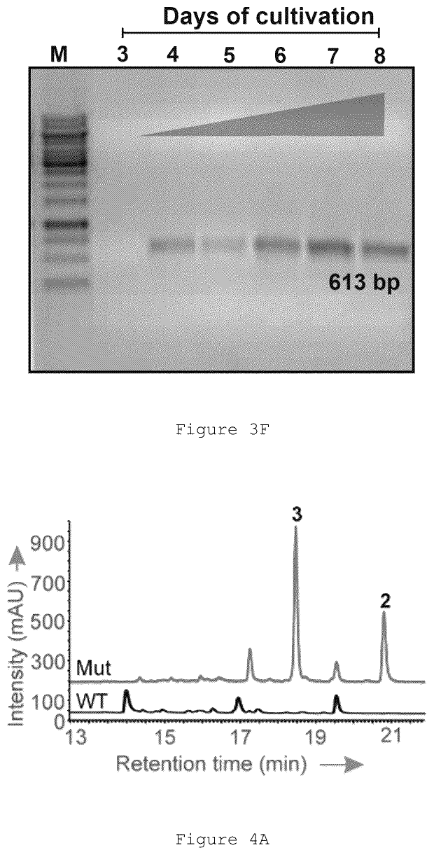

As demonstrated in FIG. 3B, mutation libraries created by the present method contain very few individual mutant cells that are negative for the fluorescent protein, which highlight the efficiency of selection in the removal of unwanted non-and low producing strains. Screening by FACS allows detection of the most promising strains from tens of millions of mutants instead of few hundreds that can be analyzed using traditional approaches in parallel cultures. Thus, the present method allows, for example, focusing strain development efforts to the most promising fraction of the mutant library.

The target product may be recovered and purified from a liquid culture of sorted metabolically engineered Actinomycetes cells by well-known methods including, but not limited to, ammonium sulphate or ethanol precipitation, affinity chromatography, anion or cation exchange chromatography, size exclusion chromatography if the target product is a protein or organic extraction, silica gel chromatography, size exclusion chromatography or high performance liquid chromatography (HPLC) if the target product is a small molecule secondary metabolite.

Biomanufacturing provides an important means for production of biomolecules for use in medicine and numerous industrial applications. The field is likely to significantly grow in the future due to advances in synthetic biology and a shift to green chemistry. The commercial feasibility and success of all of these processes depend highly on product yields obtained from bioreactors. The present metabolic engineering method presents a generally applicable technology platform for industrial strain development of Actinomycetes for production of pharmaceutical agents and industrial enzymes, where the efficiency of traditional strain development is increased by several orders of magnitude.

Accordingly, the present method for metabolic engineering can be utilized for various purposes, such as for increasing yields of proteins and secondary metabolites, to replace medium optimization, and for activation of silent BGCs in Actinomycetes strains.

Indeed, as demonstrated in the experimental part, the present method can be successfully used for drug discovery in the activation of silent metabolic pathways. More specifically, activation of a silent mutaxanthene biosynthetic gene cluster in Amycolatopsis is exemplified.

Accordingly, an embodiment of the invention relates to metabolic engineering of Amycolatopsis, more specifically to metabolic engineering of Amycolatopsis to produce at least one mutaxanthene, the method comprising:
(I′) employing a nucleic acid sequence comprising SEQ ID NO: 1 as a promoter for a mutaxanthene biosynthetic gene cluster,
(ii′) cloning the promoter into a dual reporter construct to control the expression of an antibiotic resistance gene, preferably encoding an enzyme that inactivates its target antibiotic, and a gene encoding a fluorescent protein,
(iii′) transforming cells of an Amycolatopsis strain, preferably Amycolatopsis orientalis strain, more preferably Amycolatopsis orientalis NRRL F3213 with the reporter construct,
(iv′) exposing the transformed cells to conditions that induce random mutations, thereby producing a mutant library,
(v′) transferring the mutant library into a liquid culture
(vi′) adding an antibiotic against which the antibiotic resistance gene confers resistance to the liquid culture to induce selective pressure to enrich mutants in which the transcriptional activity of the promoter has increased, thereby producing an enriched mutant library,
(vii′) fragmenting mycelia in a manner by which bacterial cell walls remain intact in the enriched mutant library to enable screening by fluorescent cell sorting, and
(viii′) screening the enriched mutant library with fragmented mycelia to obtain a metabolically engineered Amycolatopsis strain, more specifically a metabolically engineered Amycolatopsis strain that produces at least one mutaxanthene, providing a fluorescent signal, wherein the extent of the fluorescent signal correlates with the expression level of the mutaxanthene biosynthetic gene cluster, and
optionally, (ix') recovering said at least one mutaxanthene from a liquid culture of sorted metabolically engineered Amycolatopsis strain obtained in step (viii').

In an embodiment of the above method, the antibiotic resistance gene is kan or hyg. Thus, the antibiotic added to the liquid culture to induce selective pressure and to enrich the mutant library is kanamycin or hygromycin, respectively. In another embodiment of the above method, the fluorescent protein is GFP, such as sfGFP. In a further embodiment of the above method, a chemical mutagen, for example an alkylating agent such as methanesulfonate, or a physical mutagen, for example radiation such as UV, is employed to induce random mutations in step (iv′). In a still further embodiment of the above method, sonication is used to fragment mycelia in the enriched mutant library in step (vii′) in a manner by which bacterial cell walls remain intact, to enable screening by fluorescent cell sorting. In an even further embodiment of the above method, steps (iv′)-(viii′) are repeated for any number of times. It is to be understood that these embodiments can be employed independently or in any desired combinations.

The above aspect of the invention may also be formulated as a method of producing at least one mutaxanthene in an Amycolatopsis strain, preferably Amycolatopsis orientalis strain, more preferably Amycolatopsis orientalis NRRL F3213, wherein the method comprises, in this order, steps (I′)-(viii′) set forth above, optionally repeating steps (iv′)-(viii′) set forth above, and finally step (ix′) set forth above. It is to be understood that specific embodiments mentioned above for the method of metabolic engineering of Amycolatopsis to produce at least one mutaxanthene, as well as any other embodiments disclosed in this specification, apply also to the above method of producing at least one mutaxanthene.

Furthermore, the experimental part demonstrates that the present method can be successfully used for yield improvement in drug and enzyme manufacturing. This is exemplified by overproduction of cholesterol oxidase enzyme (ChoD) in Streptomyces. ChoD is a bacterial FAD-containing flavooxidase that catalyzes the first reaction in cholesterol catabolism.

Accordingly, an embodiment of the invention relates to metabolic engineering of Streptomyces, more specifically to metabolic engineering of Streptomyces to produce ChoD, the method comprising:
(I″) employing a nucleic acid sequence comprising SEQ ID NO: 2 as a promoter for the choD gene,
(ii″) cloning the promoter into a dual reporter construct to control the expression of an antibiotic resistance gene, preferably encoding an enzyme that inactivates its target antibiotic, and a gene encoding a fluorescent protein,
(iii″) transforming cells of a Streptomyces strain, preferably S. lavendulae strain with the reporter construct,
(iv″) exposing the transformed cells to conditions that induce random mutations, thereby producing a mutant library,
(v″) transferring the mutant library into a liquid culture
(vi″) adding an antibiotic against which the antibiotic resistance gene confers resistance to the liquid culture to induce selective pressure to enrich mutants in which the transcriptional activity of the promoter has increased, thereby producing an enriched mutant library,
(vii″) fragmenting mycelia in a manner by which bacterial cell walls remain intact in the enriched mutant library to enable screening by fluorescent cell sorting, and
(viii″) screening the enriched mutant library with fragmented mycelia to obtain a metabolically engineered Streptomyces strain, more specifically a metabolically engineered Streptomyces strain that produce ChoD, providing a fluorescent signal, wherein the extent of the fluorescent signal correlates with the expression level of the choD gene, and
optionally, (ix″) recovering ChoD from a liquid culture of sorted metabolically engineered Streptomyces strain obtained in step (viii″).

In an embodiment of the above method, the antibiotic resistance gene is kan or hyg. Thus, the antibiotic added to the liquid culture to induce selective pressure and to enrich the mutant library is kanamycin or hygromycin, respectively. In another embodiment of the above method, the fluorescent protein is GFP, such as sfGFP. In a further embodiment of the above method, a chemical mutagen, for example an alkylating agent such as methanesulfonate, or a physical mutagen, for example radiation such as UV, is employed to induce random mutations in step (iv″). In a still further embodiment of the above method, sonication is used to fragment mycelia in a manner by which bacterial cell walls remain intact in the enriched mutant library in step (vii″) to enable screening by fluorescent cell sorting. In an even further embodiment of the above method, steps (iv″)-(viii″) are repeated for any number of times. It is to be understood that these embodiments can be employed independently or in any desired combinations. The above aspect of the invention may also be formulated as a method of producing cholesterol oxidase in a Streptomyces strain, preferably S. lavendulae strain, wherein the method comprises, in this order, steps (I″)-(viii″) set forth above, optionally repeating steps (iv″)-(viii″) set forth above, and finally step (ix″) set forth above. It is to be understood that specific embodiments mentioned above for the method of metabolic engineering of Streptomyces to produce cholesterol oxidase, as well as any other embodiments disclosed in this specification, apply also to the above method of producing cholesterol oxidase.

The present invention also provides a metabolically engineered Actinomycetes strain obtainable by the present method or any of its embodiments. For example, provided is a metabolically engineered Amycolatopsis strain, preferably Amycolatopsis orientalis strain, more preferably Amycolatopsis orientalis NRRL F3213, which produces at least one mutaxanthene comprising a dual reporter construct, wherein an antibiotic resistance gene, preferably kan or hyg, and a gene encoding a fluorescent protein operably linked to a promoter that comprises a nucleic acid sequence of SEQ ID NO:1. As demonstrated in the examples, up to 9-fold increase in mutaxanthene yields was achieved by iterative rounds of the present method.

As another example of a metabolically engineered Actinomycetes strain obtainable by the present method, provided is metabolically engineered Streptomyces strain, preferably S. lavendulae strain, which produces cholesterol oxidase, comprising a dual reporter construct, wherein an antibiotic resistance gene, preferably kan or hyg, and a gene encoding a fluorescent protein operably linked to a promoter that comprises a nucleic acid sequence of SEQ ID NO: 2. As demonstrated in the examples, markedly increased yield of cholesterol oxidase was achieved in comparison to the parent strain that was not subjected to the present method of metabolic engineering.

### EXAMPLES

### Example 1. Materials and Methods

Reagents. All reagents were purchased from Sigma-Aldrich unless stated otherwise. All organic reagents used for HPLC and HR-MS were high-performance liquid chromatography (HPLC) grade solvents.

Strains and culture conditions. All plasmids were propagated in Escherichia coli Top10 cultured in Luria-Bertani (LB) medium with apramycin (50 μgml⁻¹) kanamycin (50 μgml⁻¹) at 37° C. E. coli ET12567/pUZ8002 was used for conjugation and grown in LB at 37° C. with appropriate antibiotics (25 μgml⁻¹ chloramphenicol, 50 μgml⁻¹ kanamycin, 50 μgml⁻¹ apramycin). To prepare spores, Streptomyces lavendulae YAKB-15 and Amycolatopsis orientalis NRRL F3213 was cultivated on mannitol-soya flour agar plates at 30° C.

Construction of reporter plasmids. Codon-optimized oligonucleotide fragment of sfgfp with strong synthetic promoter (SP44) and ribosome binding site (SR41) flanking XbaI/SpeI restriction sites (fragment I), kanamycin and hygromycin resistance genes with corresponding ribosome binding sites flanking SpeI/BamHI restriction sites (fragment II) and two fragments consisting of two strong terminator and promoter region of choD (SEQ ID NO:2) or mut (SEQ ID NO: 1) operon linked to gfp partial sequence (1-234 bp) flanked with XbaI/NdeI restriction sites (fragments III), were ordered as synthetic fragments (ThermoFisher Scientific). First pSET152 plasmid and fragment I were digested by XbaI/SpeI restriction enzymes and two purified fragments were assembled with T4 DNA ligase according to standard protocol. Then, fragment II was digested with SpeI/BamHI and ligated to the plasmid backbone to create pS_GK construct (gfp+kanamycin resistance gene). Reporter plasmids were constructed by digestion of fragment III with XbaI/NdeI and ligation to similarly cut pS_GK. The constructs were transformed into S. lavendulae and A. orientalis via conjugations.

Mutagenesis and selection. Spores (circa 10%) of S. lavendulae harboring reporter probe were added to 1.5 ml of KPO₄ (0.01 M; pH 7.0) and exposed to 200 μl ethyl methanesulfonate. The samples were vortexed for 30 seconds and incubated on shakers (300 rpm) at 30° C. for one hour, with inversions performed in 10 minute intervals. Then, the samples were centrifuged at 4000 rpm for 10 minutes at room temperature and subsequently the pellet was resuspended in one ml of freshly made and filter-sterilized 5% w/v sodium thiosulphate solution and then washed twice with one ml of H₂O and subsequently the pellet was resuspended in one ml H₂O. The homogenate was added to a 250 ml Erlenmeyer flask containing 25 ml of the antibiotic-free Y or GYM media and incubated on shaker at 300 rpm for 24 hours at 30° C. First round mutants and their controls were then sub-cultured (500 μl) in 25 ml of corresponding fresh media in three-day intervals, doubling the concentration of the kanamycin ranging from 50-400 μg/ml. Second and third round mutagenesis were conducted using the highest ChoD producing mutant in Y or GYM media resulted from first and second rounds mutagenesis, respectively. Furthermore, the second and third round mutant's kanamycin resistance was evaluated in three-day intervals by increasing kanamycin concentrations (200-900 μg/ml).

Quantitative Measurement of GFP Expression by Flow Cytometry. Mutant libraries of S. lavendulae in the Y or GYM media and A. orientalis were subjected to FACS analysis to screen mutated single cells with the highest expression of GFP. Wild type strains and strains harboring strong sfgfp was used for FACS gating. Briefly, Streptomyces mycelia (3 day-old culture in 25 ml liquid media) were harvested (4° C., 10,000×g, 5 min) and then the pellet were washed with MQ water three times and subsequently was suspended in 25 ml PBS buffer. The pellet was then subjected to ultrasonication (amplitude 20, pulse 10 s: stop 10 s, 5 min, on ice) to generate mycelia fragments. Fragments were then filtered through 5 ml Falcon® Polystyrene round bottom tubes with cell-strainer cap (Corning Science, Reynosa TAMP, Mexico). The fragmented mutated mycelia were analyzed by FACSAria Flow Cytometer with a 488-nm excitation laser and the FL1 (530/30 nm band-pass filter) detector. Each sample collected 50,000 events, and the data was analyzed by BD FACSDiva™ 8.0.2 software (BD Biosciences, CA). The parameters of the FACS setting were as follows: FSC-E00, SSC-650, FL1-400, FL2-400; threshold: FSC-50, SSC-400. The fluorescence of each sample was the geometric mean of all the measured cells and was normalized to the corresponding FSC value, which indicates the size of the cells.

Single cells were sorted in 96 well plate containing GYM media and immediately were plated in MS media. Several single colonies were streaked on MS media for three passages to guarantee obtaining pure culture. Finally mycelia transferred to Y or GYM media to evaluate ChoD production.

Cholesterol oxidase enzymatic assay. ChoD activity was measured spectrophotometrically by measuring formation of hydrogen peroxide. The stoichiometric formation of H₂O₂ during the oxidation reaction of cholesterol was monitored with ABTS at 405 nm. To determine the cell-bound ChoD, 500 μl of cultures were centrifuged at 15,000×g for 10 min. The cell pellet was resuspended in extraction buffer (0.15% Tween 80 in 50 mM phosphate buffer solution) and mixed for 30 minutes at 4° C. The suspension was centrifuged at 15,000 ×g and ChoD activity was measured from the supernatant. The activity assay mixture contained 120 μl Triton X-100 (0.05%) in 50 mM sodium-potassium phosphate buffer (pH 7), 10 μl ABTS (9.1 mM in MQ H₂O), 2.5 μl cholesterol in ethanol (1 mg/mL), 1.5 μl horseradish peroxidase solution (150 U/mL) and 20 μl of the extract preparation in a total volume of 154 μl. The spectrophotometric cholesterol activity assay was carried out in a 96-well plate. One unit of enzyme was defined as the amount of enzyme that forms 1 μmol of H₂O₂ per minute at pH 7.0 and 27° C. All the samples including sorted cells, their corresponding control cultures and wild type strain were cultured in triplicates for 24 hours in corresponding media and then subcultured into fresh media for 72 hours.

Purification of mutaxanthenes. The production strain was inoculated into 250 ml of TSB medium and grown for three days in a shaking incubator (30° C.; 300 rpm). The pre-culture was transferred to 4 L of MS-broth and cultivated for 8 days (similar growth conditions) before being harvested. The adsorbed compounds were extracted from LXA by first using 50% MeOH and then 90% MeOH. Both extracts were separately subjected to a liquid-liquid extraction by using CHCl₃ first without adjusting the pH. The neutral CHCl₃ was collected separately and the aqueous phase was further extracted with CHCl₃ after adding 1% (v/v) acetic acid. The extraction was repeated. The CHCl₃ phases were dried and saved in −20° C. The acidic CHCl₃ was subjected to a silica column (diameter 5.6 cm and length 9 cm) equilibrated with following conditions: toluene: ethyl acetate: methanol: formic acid 50:50:15:3. The first colorful front was collected in 90 ml fractions and 10 ml of ammonium actetate (1M) was added. The first four fractions were extracted using 1M ammonium acetate, the compounds of interest were in the aqueous phases. (The elution was continued after the four fractions, but the compounds of interest were in those). Next the aqueous phase was acidified using formic acid and acetic acid and extracted with CHCl₃. The CHCl₃ phases were dried, dissolved in methanol and injected to preparative HPLC.

Analytical conditions of HPLC, NMR and HRM. Active fractions were purified by HPLC on a C18 column using a linear gradient from 15% acetonitrile with 0.1% formic acid to 100% acetonitrile. The peaks of interest were collected, extracted with CHCl₃ and dried under vacuum and with nitrogen gas. NMR samples were prepared from overnight desiccated samples in CDCl₃ or MeOD. NMR analysis was performed with a 600 MHz Bruker AVANCE-III NMR-system equipped with a liquid nitrogen cooled Prodigy TCI (inverted CryoProbe) at 298 K. The experiments included 1D (¹H, ¹³C) and 2D measurements (COSY, HMBC, HSQCDE, NOESY and TOCSY). Topspin (Bruker Biospin) was used for spectral analysis. High resolution mass (MicroTOF-Q, Bruker Daltonics) was performed with direct injection of purified compounds.

Transcriptional analysis by reverse transcription analysis (RT-PCR). To assess gene expression, A. orientalis NRRL F3213 wild type and an activated mutant that were sorted by FACS were cultivated in 50 ml MS-liquid medium for 8 days. Sampling for RNA isolation was performed daily from 3 d until 8 d cultures. For this, 4 ml of the culture was harvested and gene expression was halted by addition of 444 μl stop solution (95% ethanol, 5% phenol). The mixture was incubated at room temperature for 15 min, followed by centrifugation (12,000×g; 15 min). The pellets were flash frozen in liquid nitrogen for storage at −80° C. until RNA was extracted.

Total RNA was extracted using the RNeasy Mini Kit (Qiagen) according to the manufacturer's instructions with minimal adjustments. Briefly, liquid nitrogen was employed to ground the cells to a powder. To the powder, 2 ml of phenol/CHCl₃/isoamyl alcohol (25:24:1) was added, followed by the addition of 1 ml TE (30 mM Tris-Cl, 1 mM EDTA, pH 8), and centrifuged (12,000×g; 6 min). The aqueous layer was extracted for a second time with 700 μl CHCl₃ and centrifuged (12,000×g; 4 min). To the upper layer (circa. 500 μl), 400 μl RLT and 500 μl of ethanol were added successively. The mixture was loaded onto the column, rinsed with 350 μl RW1, and then 80 μl of DNase I digest was applied to the column, followed by incubation at room temperature for 30 min before being washed with RW1 (350 μl) and RPE (500 μl; 2 times). Eventually, the column was eluted with 80 μl RNase-free water. The extracted mRNA was checked for purity on a 0.8% agarose gel and stored at −80° C. until further use.

Synthesis of cDNA from the extracted mRNA was performed using the ThermoScientific RevertAid First Strand cDNA synthesis kit with modest adjustments. Briefly, the 12 μl reaction contained 4 μg of template RNA, 1 μl of random hexamer primer, and DEPC water. This reaction mixture was incubated at 65° C. for 5 min, before being cooled on ice. To this mixture, 5× reaction buffer (4 μl), RiboLock RNase inhibitor (20 U/μl-1; 1 μl), 10 mM dNTP mix (2 μl) and reverse transcriptase (200 U/μl-1; 1 μl) was added. The reaction mixture was incubated at 25° C. for 5 min, then at 42° C. for 65 min before being terminated by heating at 70° C. for 5 min.

RT-PCR amplification of the expressed gene targeting to the minimal polyketide synthase II gene regions was performed using the complementary DNA (CDNA) as a template and a pair of degenerate primers (5′-TSGCSTGCTTGGAYGCSATC-3′; SEQ ID NO: 3) (sense primer) and (5′-TGGAANCCGCCGAABCCGCT-3′; SEQ ID NO: 4) to amplify a product with the size of 613 bp. PCR product was analyzed by gel electrophoresis on a 0.8% agarose gel stained with SybrSafe and compared to a one kb Plus Ladder (Invitrogen). RT-PCR amplifications was performed for all the samplings harvested, viz., from 3d until 8d for both wild type and the mutants. The 50 μl of the reaction mixture for the RT-PCR amplification contained cDNA (1000× diluted): 1 μl; 10×GC buffer: 10 μl; 10 mM dNTP mix: 2.5 μl; DMSO: 2.5 μl; Primers (2.5 μl each for forward and reverse); Dream Taq DNA polymerase: 0.5 μl; and DEPC water: 28.5 μl. The thermocycling conditions comprise an initial longer denaturation phase (96° C.; 2 min). The cycle steps for 30 cycles were as follows: denaturation (96° C.; 1 min), annealing (58° C.; 2 min), and extension (73° C.; 1.5 min). The reaction was terminated with a longer final extension (73° C.; 8.5 min). RT-PCR was performed on a SureCyler 8800 (Agilent Technologies, Santa Clara, California, USA).

### Example 2. Construction of a Double Reporter System

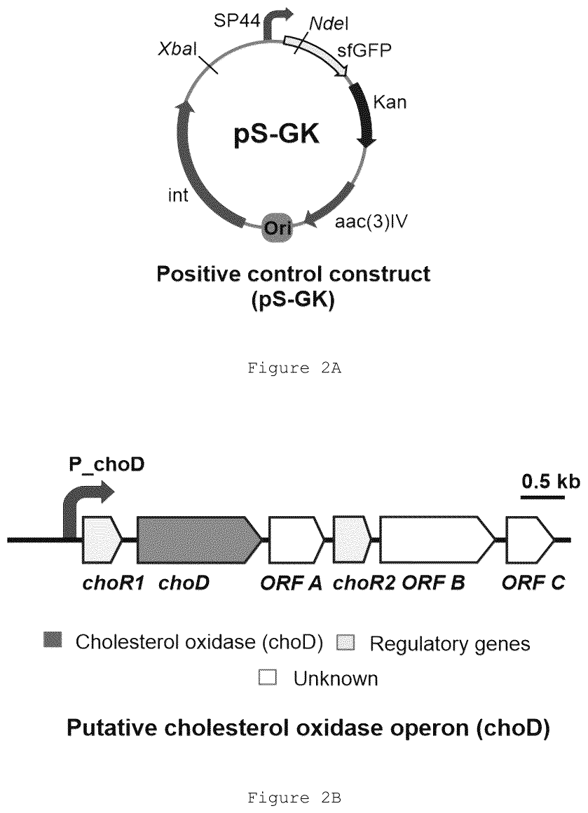

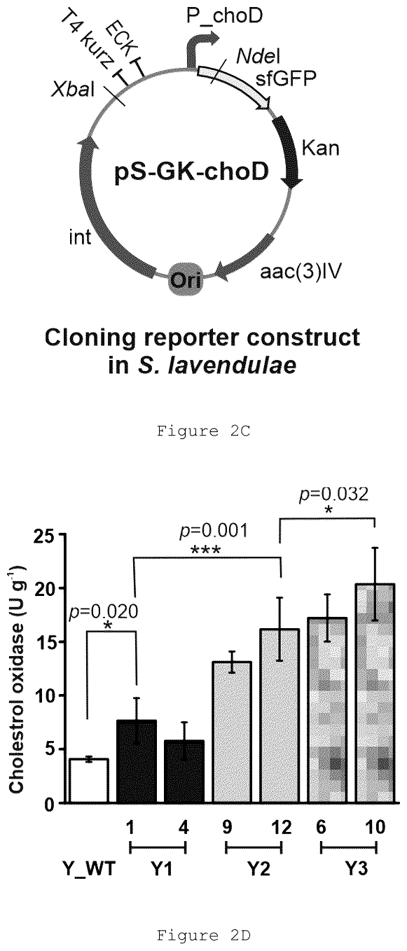

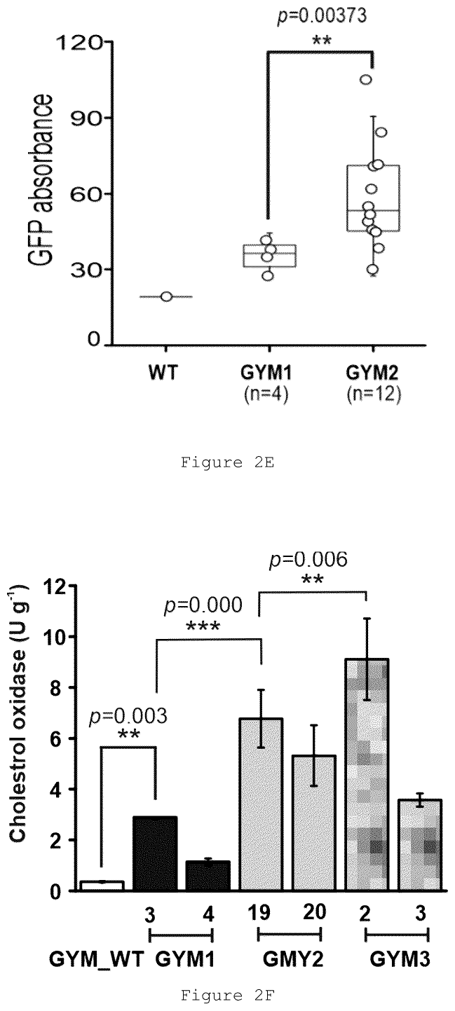

In order to convert Actinomycetes strain develop into single cell format, the inventors constructed a double reporter gene system in the integrative single copy pSET152 vector. The inventors opted for two alternative antibiotic resistance marker genes, kan and hyg to impose selection using either kanamycin and hygromycin, respectively. Importantly, both kan and hyg encode enzymes that inactivate their target antibiotics (aminoglycoside acyl transferase and hygromycin phosphotransferase, respectively), instead of modify the drug targets. This allowed the inventors to link the transcription level of the resistance gene to survival of the bacterial strain under elevated concentrations of antibiotics. For the second reporter gene, the inventors chose superfolder Green Fluorescent Protein (sfGFP) to allow screening by FACS. The reporter genes were insulated to avoid promoter leakage due to transcription from upstream genes (i.e. bacteriophage phi31 integrase and apramycin resistance genes) using two strong terminator sequences, a synthetic T4 kurz and a natural terminator ECK120029600. As a positive control, the inventors devised plasmid pS-GK (FIG. 2A), where the reporter genes are cloned under the strong synthetic promoter SP44.

### Example 3. Development of SCMS for Protein Overproduction

First the inventors focused on production of a cholesterol oxidase enzyme ChoD by an industrial strain S. lavendulae YAKB-15. The promoter sequence (SEQ ID NO: 2) of the operon including choD (FIG. 2B) was cloned into the reporter construct in Escherichia coli and the resulting plasmid ps_GK_ChoD (FIG. 2C) was introduced to S. lavendulae YAKB-15 by intergeneric conjugation. The exconjugants could withstand kanamycin concentrations up to 50 ug/mL in the production medium, reflecting the natural transcriptional level of choD in the strain. Next the inventors generated a mutant library of S. lavendulae YAKB-15/pS_GK_Chod by treatment of spores with 200 ul ethyl methanesulfonate with an approximate kill rate of 99%. The spores were harvested and the library was used to inoculate parallel 25 mL cultivations in Y production medium supplemented with varying concentrations (50 ug/ml-400 ug/ml) of kanamycin, with the objective of reducing the library size by enriching best performing mutants and killing off undesired low or non-producing mutants. Bacterial growth could still be observed in cultures with 400 ug/ml kanamycin, which demonstrated an eight-fold increase in the tolerance of the mutant library towards the antibiotic.

FACS is an ideal method for sorting heterogeneous mixtures of biological cells, but has not been widely adopted for Actinomycetes. A key problem is that the nozzle size of the instruments (70 uM) is typically orders of magnitude smaller than growing Streptomyces mycelium (e.g. 260-950 uM). This was circumvented in pioneering work by using protoplasts to pass through the instrument. However, the method has not gained wide use, possibly because of the fragility of protoplasts and very low survival rates in cell sorting. Here the inventors opined that fragmentation of the mycelium by sonication might be sufficient to allow the cells to pass through the nozzles. Coupled with sample filtration and careful instrument gating, this simple method allowed the inventors to analyze Streptomyces cells by FACS.

The methodology development allowed the inventors to screen approximately 20 million mutants from the enriched S. lavendulae YAKB-15/pS_GK_Chod library to find individual cells with the highest sfGFP fluorescence. Positive mutants were harvested and analyzed for cholesterol oxidase activity in flask cultures in Y production medium supplemented with 400 ug/mL kanamycin (FIG. 2d). The mutant Y1_1 displayed a near two-fold increase in yield of ChoD (7.65 U/g) in comparison to the wild type (4.07 U/g).

Next the inventors concluded that the methodology could be used in an iterative manner and performed two more rounds of SCMS using the best performing mutant from the previous round as starting material. Single cells derived from iterative second and third round mutagenesis and selection were resistant to 800 ug/mL and 900 ug/mL kanamycin, respectively. During these experiments the inventors noted fluctuations in the production profiles of the strains, which the inventors surmised might be due to acquisition of small clusters of cells, instead of single cells, by FACS. Product yields were stabilized by acquisition of pure cultures by streak plating and preliminary fluorometer analysis of cultures to find best performing mutants (FIG. 2E). In total, three rounds of SCMS resulted in a five-fold yield enhancement in Y3_10 (20.35 U/g) in comparison to S. lavendulae YAKB15.

### Example 4. Application of SCMS to Replace Medium Optimization

The production of cholesterol oxidase ChoD is tightly regulated in S. lavendulae YAKB-15 and is linked to the presence of whole yeast cells in the Y culture medium. Establishing optimal culture conditions is a necessity for industrial production of natural products and proteins by Actinomycetes, since production levels fluctuate greatly in response to changing conditions. Since this significantly complicates media optimization, the inventors surmised that the ability to take advantage of selection in strain development could be used to change the paradigm: instead of finding optimal conditions for high yield production, SCMS could be used to find best performing mutants under preset culture conditions. The inventors chose simple yeast extract based GYM medium, where wild type S. lavendulae YAKB-15 produced low amounts of ChoD (0.36 U/g). Three rounds of single cell mutant selection led to continuously improved product yields (FIG. 2f) and generation of a strain with 25-fold increased ChoD yield in GYM3_2 (9.11 U/g). The mutant libraries were found to be resistant to 200 ug/mL, 400 ug/mL and 600 ug/mL kanamycin in the first, second and third round of mutation, respectively, demonstrating the importance of the enrichment step.

### Example 4. Use of SCMS for Activation of Silent Metabolic Pathways

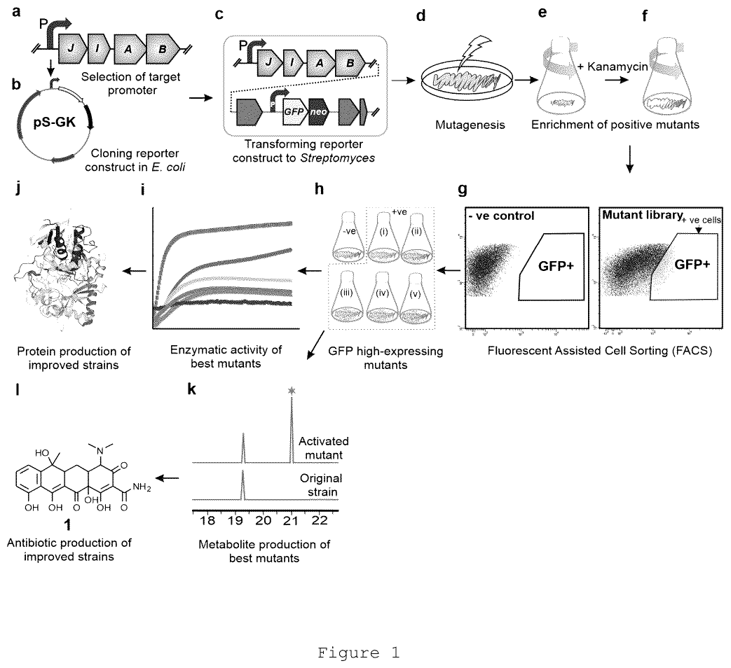

Next the inventors turned their attention to small molecule microbial natural products. The inventors mined the genomes of selected Actinomycetes strains for the presence of BGCs encoding aromatic type II polyketides. A tetracycline-type BGC (FIG. 3a) was detected in a strain categorized as Amycolatopsis orientalis NRRL F3213 based on Average Nucleotide Identity (ANI). Sequence analysis of the BGC revealed a set of core genes encoding ketosynthase (KS) α and β subunits, acyl carrier protein (ACP) and dedicated cyclases (CYC) that were similar to those found on the biosynthetic pathway of tetracycline (1, FIG. 1i). In contrast to the core genes, other regions of the BGC in A. orientalis NRRL F3213 harbored notable differences to all metabolic pathways deposited in databases.

The inventors examined cultures of wild type A. orientalis NRRL F3213 for the presence of pigmented metabolites similar to tetracyclines by HPLC-UV/Vis, but since none could be detected, the inventors concluded that the pathway might be silent. The inventors proceeded to clone the promoter region of an operon controlling the expression of the SARP-family regulatory gene and the essential, translationally coupled, KS_{α} and KS_{β} responsible for synthesis of the polyketide scaffold (FIG. 3A), to generate the activation construct pSGKP45 in E. coli. Introduction of the plasmid to A. orientalis NRRL F3213 did not induce formation of a sfGFP signal, reinforcing our hypothesis that the BGC was not transcribed under laboratory conditions. In order to activate the metabolic pathway, the inventors proceeded to carry out mutagenesis of spores by UV radiation, enrichment under kanamycin selection (200 ug/mL) and cell sorting by FACS (FIG. 3B). Single cells with positive sfGFP signal were harvested and grown in liquid cultures and on plates (FIG. 3C), which revealed that the mutants were phenotypically distinct from the wild type and produced dark pigmented metabolites. In order to validate that the targeted pathway had been activated in the mutant, the inventors performed transcription analysis by RT-PCR (FIG. 3D-3F). Amplification of a fragment from the KS. gene confirmed that the BGC was active in A. orientalis NRRL F3213/pSGKP45_UV1 (3), but remained silent in the wild type (FIG. 3E). Time-course analysis indicated that the activation occurred on day four and continued until end of the cultivation period on day eight in the mutant strain (FIG. 3F).

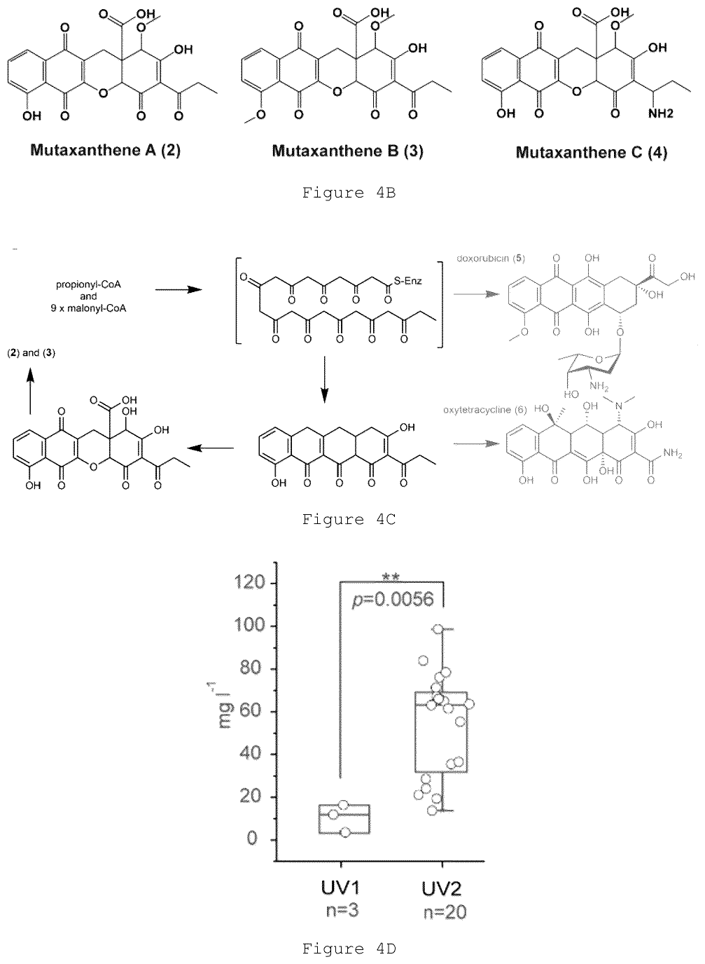

Comparative metabolic analysis of culture extract by HPLC-UV/Vis (FIG. 4A) revealed that the mutated strain A. orientalis NRRL F3213/pSGKP45_UV1 (3) produced two main secondary metabolites 2 and 3 (FIG. 4b) that were not synthesized by the parental wild type strain. Furthermore, the inventors noted that 2 was converted to 4 (FIG. 4B) during extractions in the presence of ammonium acetate buffer. The mutant strain was cultivated in large-scale to obtain sufficient material for structure elucidation by NMR and HR-MS. The NMR data confirmed that 2, 3 and 4 were known polyketide natural products denoted as mutaxanthenes A, B and D, respectively Conversion of mutaxanthenes to their eneamine congeners in pH ˜6. 6 ammonium acetate buffer have been noted previously.

### Example 5. Biosynthesis of Mutaxanthenes

Analysis of the BGC provided further confirmation that the activated pathway is responsible for mutaxanthene biosynthesis. The biosynthesis is initiated from a propionyl-CoA starter unit and nine malonyl-CoA extender units that lead to formation of an unreduced decaketide (FIG. 4C), similarly to the metabolic pathway of the anticancer agent doxorubicin (5). The close relationship to oxytetracycline (6) biosynthesis was revealed by the presence of conserved cyclases that determine the folding pattern of the polyketide. Deviations from the oxytetracycline paradigm are due to additional genes that encode FAD-dependent proteins. These redox enzymes are likely to be responsible for formation of the multiple skeletal rearrangements required for biosynthesis of mutaxanthenes.

### Example 6. Improving Yields of Mutaxanthenes by SCMS

The structures of 2 and 4 differ only in regards to 7-O-methylation and the combined carbon flux to mutaxanthenes was calculated to be 11 mg/L in the best performing first round mutant A. orientalis NRRL F3213/pSGKP45_UV1 (3). In order to improve production, the inventors performed a second round of SCMS and acquired 40 pure culture mutants to estimate the robustness of the methodology. The number of mutants was first reduce to 20 by ranking them based on fluorescence of the cultures and the metabolic profiles of these strains were estimated by HPLC-UV/Vis. The second round strains displayed significantly increased yields with an average five-fold improved production (FIG. 4D). The best performing mutant A. orientalis NRRL F3213/pSGKP45_UV2(38) displayed nine-fold increased yields (99 mg/L) in comparison to the parental strain.

It is obvious to a person skilled in the art that with the advancement of technology, the basic idea of the invention may be implemented in various ways. The invention and its embodiments are thus not limited to the examples described above, instead they may vary within the scope of the claims.

1. A method for metabolic engineering of Actinomycetes, wherein the method comprises in this order:
(i) identifying and selecting a promoter from a target gene or a gene involved in biosynthesis of a target product,
(ii) cloning the promoter into a dual reporter construct to control the expression of a first reporter gene which is an antibiotic resistance gene and a second reporter gene encoding a fluorescent protein,
(iii) transforming an Actinomycetes strain with the reporter construct,
(iv) exposing the transformed Actinomycetes strain to conditions that induce random mutations, thereby producing a mutant library,
(v) transferring the mutant library into a liquid culture,
(vi) adding an antibiotic corresponding to the antibiotic resistance gene to the liquid culture to induce selective pressure to enrich mutants in which transcriptional activity of the selected promoter has increased, thereby producing an enriched mutant library,
(vii) fragmenting mycelia in a manner by which bacterial cell walls remain intact in the enriched mutant library to enable screening by fluorescent cell sorting, and
(viii) screening the mutant library with fragmented mycelia by fluorescent cell sorting to obtain a metabolically engineered Actinomycetes strain that provides a fluorescent signal, wherein the an extent of the fluorescent signal correlates with the an expression level of the target gene or the gene involved in the biosynthesis of the target product.

2. The method according to claim 1, wherein steps (iv)-(viii) are repeated for any number of rounds.

3. The method according to claim 1, further comprising step (ix), wherein an expression product of the target gene, or the target product is recovered from a liquid culture of sorted metabolically engineered Actinomycetes strain obtained in step (viii).

4. The method according to claim 1, wherein step (vii) comprises fragmenting the mycelia by sonication.

5. The method according to claim 4, wherein the sonication is ultrasonication.

6. The method according to claim 1, wherein the antibiotic resistance gene encodes an enzyme that inactivates its target antibiotic.

7. The method according to claim 6, wherein the enzyme is an aminoglycoside acyltransferase or hygromycin phosphotransferase.

8. The method according to claim 1, wherein the antibiotic resistance gene is kan or hyg.

9. The method according to claim 1, wherein the dual reporter construct further comprises a selective marker, preferably an antibiotic resistance gene different from the first reporter gene, operably linked to an appropriate promoter to enable propagation of the dual reporter construct in a bacterial host other than the Actinomycetes to be metabolically engineered.

10. The method according to claim 9, wherein the bacterial host is E. coli.

11. The method according to claim 9, wherein the bacterial host is used for transforming the Actinomycetes strain by conjugation.

12. The method according to claim 1, wherein the Actinomycetes strain is an Amycolatopsis strain, preferably Amycolatopsis orientalis strain, more preferably Amycolatopsis orientalis NRRL F3213, the target product is a mutaxanthene and the promoter of step (i) comprises a nucleic acid sequence depicted in SEQ ID NO: 1.

13. The method according to claim 1, wherein the Actinomycetes strain is a Streptomyces strain, preferably S. lavendulae strain, the target product is a cholesterol oxidase and the promoter of step (i) comprises a nucleic acid sequence depicted in SEQ ID NO: 2.

14. A metabolically engineered Actinomycetes strain obtainable by the method according to claim 1.

15. Use of the method according to claim 1 for producing or increasing yield of an endogenous target protein or secondary metabolite, for replacing medium optimization in the production of a target protein or secondary metabolite, and for activating a silent target gene or a silent biosynthetic gene cluster.

16. A metabolically engineered Amycolatopsis strain which produces at least one mutaxanthene, comprising a dual reporter construct, wherein an antibiotic resistance gene, preferably kan or hyg, and a gene encoding a fluorescent protein operably linked to a promoter that comprises a nucleic acid sequence of SEQ ID NO:1.

17. A metabolically engineered Streptomyces strain which produces cholesterol oxidase, comprising a dual reporter construct, wherein an antibiotic resistance gene, preferably kan or hyg, and a gene encoding a fluorescent protein operably linked to a promoter that comprises a nucleic acid sequence of SEQ ID NO:2.


SEQUENCE LISTING (4 sequences)

SEQ ID NO: 1 - DNA, 167 bp, synthetic construct
  source 1..167: mol_type = other DNA
tccgtccgcgcgtacggccagcggtcccaatattgcgatctgcatccgacccccagatacctgccgaaagagcacgccaggcagctgcccgcgtgcttcgctttcccccagtgagacgccagcgacgctagcagagaaacggcttcctgatcccgggcgggaaaaac

SEQ ID NO: 2 - DNA, 440 bp, synthetic construct
  source 1..440: mol_type = other DNA
gggccccagcaccctgaggaggggctcggcccggccgctggtcatgggcgacgtgttcacgggaggcgtggacatgacggtccccgatctcttacgggtacggctggttgacggacggcttcgcgcaggggctgtcgcagggcgtccgctaccgatcatgacatagcgaaagcgcctgctgagctggcgatccgtcgattggcaagaaagcagaaggtgttgggcaaggatttcgacaaggggtccggaaaacgacggcagcgggcggcaaggggacgtcagcgactcgaccaggaccccgcaaggtcggtgacgcaggatgtccgggtctggagatactgctggtccagggccgatgacgcccctgctgacggcctccccggccgctcaccagcctccgaattgatttcgcgctcaagtcttcgacagggagcccattc

SEQ ID NO: 3 - DNA, 20 bp, synthetic construct
  source 1..20: mol_type = other DNA
tsgcstgcttggaygcsatc

SEQ ID NO: 4 - DNA, 20 bp, synthetic construct
  source 1..20: mol_type = other DNA
tggaanccgccgaabccgct